(19) United States
(12) Patent Application Publication
(10) Pub. No.: US 2025/0171408 A1
(43) Pub. Date: May 29, 2025

(54) NOVEL SOLID STATE FORMS OF INDOXACARB

(71) Applicant: ADAMA MAKHTESHIM LTD., Beer Sheva (IL)

(72) Inventors: Jayapal Reddy BICIDI, Bayyaram (IN); Amaresh RAMIYA RAJARAM, Hyderabad (IN); Bijukumar Gopinathan PILLAI, Hyderabad (IN); Hagit FORCKOSH, Beer Sheva (IL); Chen VAZANA, Ramat-Gan (IL); Natalia STRIZHIVER, Lehavim (IL)

(73) Assignee: ADAMA MAKHTESHIM LTD., Beer Sheva (IL)

(21) Appl. No.: 18/834,307

(22) PCT Filed: Feb. 2, 2023
(86) PCT No.: PCT/IL2023/050118

§ 371 Date: Jul. 30, 2024

(30) Foreign Application Priority Data
Feb. 2, 2022 (IN) 202211005633

(51) Int. Cl. C07D 273/02; A01N 47/38; A01P 7/04

(52) U.S. Cl. CPC C07D 273/02; A01N 47/38; A01P 7/04

(57) ABSTRACT

The present disclosure relates to solid state forms of Indoxacarb or its solvate, processes for preparation thereof and agrochemical compositions thereof.

### TECHNICAL FIELD

The present disclosure relates to new solid state forms of Indoxacarb or its solvate, processes for preparation thereof and agrochemical compositions thereof.

### BACKGROUND OF THE DISCLOSURE

The control of various pests is extremely important in achieving high crop efficiency. Damage by insects such as lepidopteron pests to growing and stored agronomic crops can cause significant reduction in productivity and thereby result in increased costs to the consumer. Certain oxadiazines, such as Indoxacarb and compositions thereof are suitable for agronomic and non-agronomic uses, and they are used for controlling lepidopteron pests in both agronomic and non-agronomic environments. The control of lepidopteron pests in forestry, greenhouse crops, ornamentals, nursery crops, stored food and fibre products, livestock, household, and public and animal health is also important.

Indoxacarb is an oxadiazine insecticide which is active against a wide spectrum of insect pests from at least ten orders and well over thirty families. Lepidopteran insects represent the majority of pests on the Indoxacarb label, but it is also used to control many species of plant bugs, leafhoppers, flea hoppers, weevils, beetles, flies, cockroaches, and ants.

Indoxacarb has the chemical name, methyl (4aS)-7-chloro-2-[methoxycarbonyl-[4-(trifluoromethoxy)phenyl]carbamoyl]-3,5-dihydroindeno[1,2-e][1,3,4]oxadiazine-4a-carboxylate.

Indoxacarb has the following chemical structure:

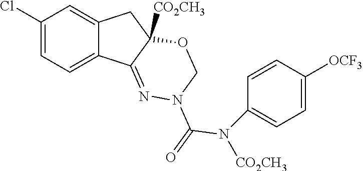

Arthropodicidal oxadiazines and the corresponding synthetic methods for the preparation of biologically active oxadiazines are previously disclosed in PCT patent applications WO 92/11249 and WO 93/19045. Methods of preparing these compounds have also been reported in WO 95/29171.

Different polymorphic forms of Indoxacarb of different isomeric purities have been reported in the literature such as in CN103694193; BR102017023281; WO2021005545 and WO2021005549 patent documents.

Furthermore, another PCT publication, WO2018178982 discloses preparation of a solid state form of Indoxacarb which is racemic or enantiomerically enriched at chiral centre. These publications have been incorporated in this application as references.

Polymorphism, the occurrence of different crystal forms, is a property of some molecules and molecular complexes. A single compound, like Indoxacarb or its solvate, may give rise to a variety of polymorphs having distinct crystal structures and physical properties like melting point, thermal behaviours (e.g., measured by thermogravimetric analysis—“TGA”, or differential scanning calorimetry—“DSC”), X-ray powder diffraction (XRPD) pattern, infrared absorption fingerprint, Raman absorption fingerprint, and solid state (¹³C-) NMR spectrum. One or more of these techniques may be used to distinguish different polymorphic forms of a compound.

Different solid state forms of an active ingredient or its solvate (of different optical isomeric purities) may possess different properties. Such variations in the properties of different isomers and solid state forms may provide a basis for improving formulation, for example, by facilitating better processing or handling characteristics, improving the dissolution profile, or improving stability (polymorph as well as chemical stability) and shelf-life. These variations in the properties of different salts and solid state forms may also provide improvements to the final formulation or recipe, for instance, if they serve to improve dissolution. Different isomers and solid state forms of an active ingredient or its solvate may also give rise to a variety of polymorphs or crystalline forms, which may in turn provide additional opportunities to use variations in the properties and characteristics of a solid active ingredient for providing an improved product.

Discovering new solid state forms and optical isomeric purity of an agrochemical product can provide materials having desirable processing properties, such as ease of handling, ease of processing, storage stability, and ease of purification or as desirable intermediate crystal forms that facilitate conversion to other polymorphic forms. New polymorphic forms of an active ingredient can also provide an opportunity to improve the performance characteristics of an agrochemical product (dissolution profile, permeability, etc.). It expands the range of materials that an expert in the field can avail for formulation optimization, for example by providing a product with different properties, e.g., a different crystal habit, higher crystallinity or polymorphic stability which may offer better processing or handling characteristics, improved dissolution profile, or improved shelf-life.

For at least these reasons, there is a need for new solid state forms (including new optical isomeric purity) of Indoxacarb or its solvates.

### SUMMARY

The present disclosure relates to new solid state forms of Indoxacarb or its solvate, processes for preparation thereof, and agrochemical compositions including this solid state forms.

The present disclosure also provides use of the solid state forms of Indoxacarb or its solvate for preparing other solid state forms of Indoxacarb, and solid state forms of Indoxacarb in certain optical isomeric mixture.

In an embodiment, the present disclosure provides use of Indoxacarb Form A for preparing new Form B of Indoxacarb.

In another embodiment, the present disclosure provides use of Indoxacarb Form A for preparing new Form I of Indoxacarb isopropanolate solvate.

The present disclosure also provides solid state form of Indoxacarb for uses in the preparation of other solid state forms of Indoxacarb or its solvate, and solid state form of Indoxacarb in certain optical isomeric mixture.

In one embodiment, the present disclosure encompasses the described solid state forms of Indoxacarb or its solvates for use in the preparation of agrochemical compositions and/or formulations, optionally for agronomic and non-agronomic uses, and a method of their use for controlling lepidopteron pests in both agronomic and non-agronomic environments.

In another embodiment, the present disclosure encompasses uses of the described solid state forms of Indoxacarb or its solvates for the preparation of agrochemical compositions and/or formulations.

The present disclosure further provides agrochemical compositions including one or more solid state forms of Indoxacarb or its solvates according to the present disclosure.

In yet another embodiment, the present disclosure encompasses agrochemical formulations including one or more of the described solid state forms of Indoxacarb or it solvate and at least one additional component selected from the group consisting of a surfactant, a solid diluent and liquid diluent and optionally an effective amount of at least one additional biologically active compound or agent.

The present disclosure encompasses processes to prepare said agrochemical formulations of Indoxacarb or its solvates including one or more of the described solid state forms and at least one additional component selected from the group consisting of a surfactant, a solid diluent and liquid diluent and optionally an effective amount of at least one additional biologically active compound or agent.

The solid state forms defined herein as well as the agrochemical compositions or formulations of the solid state forms of Indoxacarb or its solvates can be used as oxadiazine insecticide for controlling lepidopteron pests in both agronomic and non-agronomic environments.

The present disclosure also provides methods for controlling lepidopteron pests comprising applying solid state form of Indoxacarb or its solvates by blocking the sodium channels in the pest's nervous system, entering through the stomach and contact routes.

The present disclosure also provides uses of the solid state forms of Indoxacarb or its solvates of the present disclosure, or at least one of the above agrochemical compositions or formulations for controlling lepidopteron pests in both agronomic and non-agronomic environments.

### BRIEF DESCRIPTION OF THE FIGURES

FIG. 1 shows an X-ray powder diffractogram (XRPD) of Form B of Indoxacarb.

FIG. 2 shows Differential scanning calorimetry (DSC) graph of Form B of Indoxacarb.

FIG. 3 shows an X-ray powder diffractogram (XRPD) of Form A of Indoxacarb, in accordance with the process of PCT publication, WO2018178982.

FIG. 4 shows Differential scanning calorimetry (DSC) graph of Form A of Indoxacarb, in accordance with the process of PCT publication, WO2018178982.

FIG. 5 shows an X-ray powder diffractogram (XRPD) of Form I of Indoxacarb isopropanolate solvate.

FIG. 6 shows Differential scanning calorimetry (DSC) graph of Form I of Indoxacarb isopropanolate solvate.

### DETAILED DESCRIPTION

The present disclosure relates to solid state forms of Indoxacarb or its solvates, processes for preparation thereof and agrochemical compositions including the solid state form. The disclosure also relates to the conversion of the described a solid state form of Indoxacarb to other solid state forms of Indoxacarb or its solvates.

The solid state forms of Indoxacarb or its solvates according to the present disclosure may have advantageous properties selected from at least one of: chemical or polymorphic purity, flowability, solubility, dissolution rate, morphology or crystal habit, stability—such as chemical stability as well as thermal and mechanical stability with respect to polymorphic conversion, stability towards dehydration and/or storage stability, a lower degree of hygroscopicity, low content of residual solvents and advantageous processing and handling characteristics such as compressibility, or bulk density.

A crystal form may be referred to herein is being characterized by graphical data as shown in a Figure. Such data include, for example, powder X-ray diffractograms and DSC data. In an embodiment of this invention powder X-ray diffractograms of Indoxacarb crystal may show peaks for predominant optical isomer (S-isomer) along with some additional peaks of racemic Indoxacarb crystal which might have been formed in minor quantity because of the presence of minor optical isomer (R-isomer).

As is well-known in the art, the graphical data potentially provides additional technical information to further define the respective solid state forms which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Indoxacarb referred to herein as being characterized by graphical data “as shown in” a Figure will thus be understood to include any crystal form of Indoxacarb or its solvates, characterized with the graphical data having such small variations, as are well known to the skilled person, in comparison with the Figure.

A pure solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% or less, about 10% or less, about 5% or less, about 2% or less, about 1% or less, or 0% of any other forms of the subject compound as measured, for example, by XRPD. Thus, the pure solid state forms of Indoxacarb or its solvates may be described herein as substantially free of any other solid state forms would be understood to contain greater than about 80% (w/w), greater than about 90% (w/w), greater than about 95% (w/w), greater than about 98% (w/w), greater than about 99% (w/w), or 100% of the subject solid state form of Indoxacarb. Accordingly, in some embodiments of the disclosure, the described pure respective solid state forms of Indoxacarb or its solvate may contain from about 1% to about 20% (w/w), from about 5% to about 20% (w/w), or from about 5% to about 10% (w/w) of one or more other solid state forms of the same Indoxacarb.

In another aspect, the present disclosure contemplates that a certain solid state form of Indoxacarb can exist in the presence of any other of the solid state forms or mixture thereof. Accordingly, in one embodiment, the present disclosure provides form B, for example, wherein form B is present in a solid form that includes less than 95%, less than 90%, less than 80%, less than 70%, less than 60%, less than 50%, less than 40%, less than 30%, less than 20%, less than 10%, less than 5%, less than 3% or less than 1% by weight of any other physical forms of Indoxacarb. Likewise, in another embodiment, the present disclosure provides Form I, for example, wherein form I of Indoxacarb isopropanolate is present in a solid form that includes less than 95%, less than 90%, less than 80%, less than 70%, less than 60%, less than 50%, less than 40%, less than 30%, less than 20%, less than 10%, less than 5%, less than 3% or less than 1% by weight of any other physical forms of Indoxacarb.

The solid state forms of Indoxacarb or its solvate as characterized herein may be present also in traces in an agrochemical compositions or formulations for controlling lepidopteron pests in both agronomic and non-agronomic environments.

As used herein, unless stated otherwise, XRPD peaks reported herein are optionally measured using CuK_{α}, radiation, λ=1.54187 Å.

As used herein, the term “isolated” in reference to solid state forms of Indoxacarb or its solvate of the present disclosure corresponds to solid state form of Indoxacarb that is physically separated from the reaction mixture in which it is formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., about 22° C. to about 27° C., or about 25° C.

A process or step may be referred to herein as being carried out “overnight.” This refers to a time interval, e.g., for the process or step, that spans the time during the night, when that process or step may not be actively observed. This time interval is from about 8 to about 20 hours, about 10 to about 18 hours, or about 16 hours.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding methyl tert-butyl ether (MTBE) (1.5 v/v) to a 100 ml reaction mixture would indicate that 150 mL of MTBE was added.

As used herein, the term “reduced pressure” refers to a pressure of about 10 mbar to about 50 mbar.

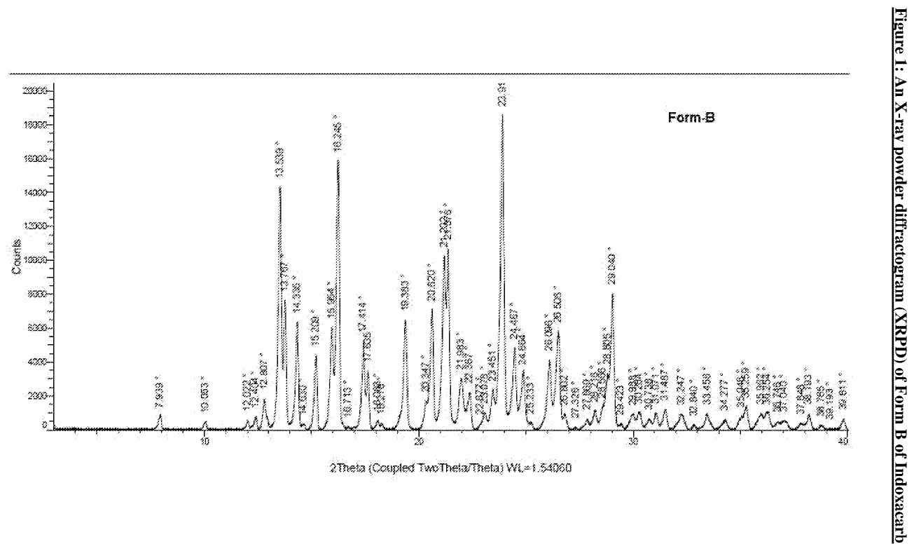

The present disclosure includes a solid state form of Indoxacarb designated as Form B. The Form B of Indoxacarb may be characterized by data selected from one or more of the following: an XRPD pattern having peaks at 13.5, 13.8, 16.2, 21.2, and 21.4 degrees 2-theta±0.2 degrees 2-theta; an XRPD pattern substantially as shown in FIG. 1; and combinations of these data, wherein by “substantially” it is meant that the reported peaks can vary by about ±0.2°. It is well known in the field of XRPD that while relative peak heights in spectra are dependent on a number of factors, such as sample preparation and instrument geometry, peak positions are relatively insensitive to experimental details.

Crystalline Form B of Indoxacarb may be further characterized by the XRPD pattern having peaks at 13.5, 13.8, 16.2, 21.2, and 21.4 degrees 2-theta±0.2 degrees 2-theta, and also having one, two or three additional peaks selected from 7.9, 19.4 and 23.9 degrees 2-theta±0.2 degrees 2-theta. Crystalline Form B of Indoxacarb may be characterized by any combination of at least four peaks in an XRPD pattern substantially as shown in FIG. 1. Crystalline Form B of Indoxacarb may be further characterized by FIG. 1, wherein some additional peaks, for example, at 12.0, 12.4 and 12.8 degrees 2-theta may be attributed to other minor form(s) of Indoxacarb.

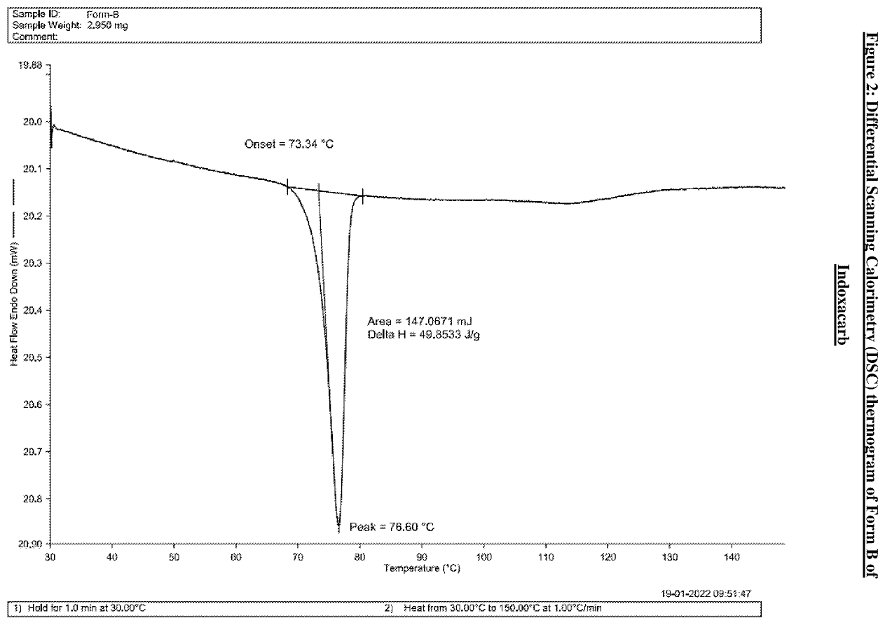

Crystalline Form B of Indoxacarb also exhibits a Differential Scanning Calorimetry (DSC) thermogram substantially as shown in FIG. 2, which is characterized by a predominant endotherm peak at about between 70-80° C. as measured by Differential Scanning Calorimeter at a scan rate of 1° C. per minute. Crystalline Form B of Indoxacarb is anhydrous.

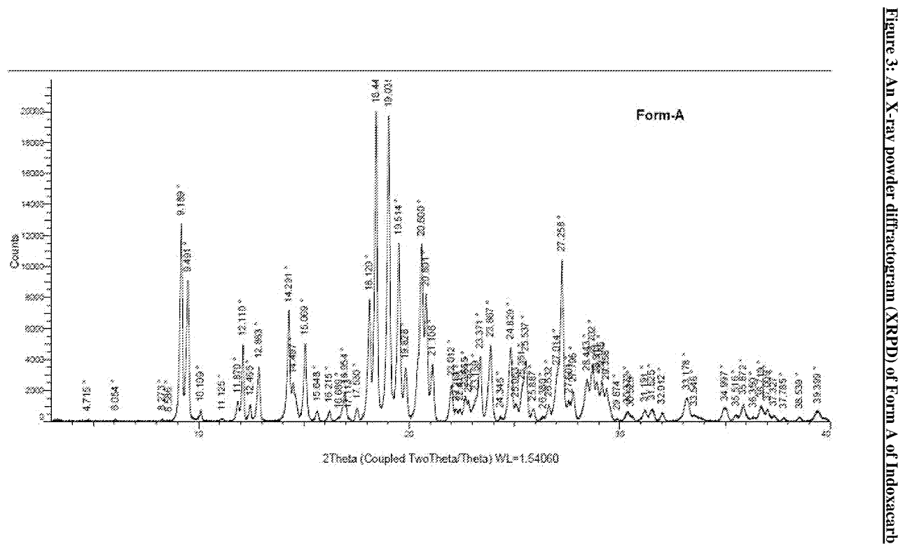

The reference solid state Form A of Indoxacarb prepared following the process of the PCT publication WO2018178982, may be characterized by data selected from one or more of the following: an XRPD pattern having peaks at 9.2, 9.5, 12.1, 12.9, 14.3, 15.1, 18.1, 18.4, 19.0, 19.5, 20.6, 20.8, 23.4, 23.9, 24.8 and 27.3 degrees 2-theta±0.2 degrees 2-theta; an XRPD pattern substantially as shown in FIG. 3; and combinations of these data, wherein by “substantially” it is meant that the reported peaks can vary by about ±0.2°. It is well known in the field of XRPD that while relative peak heights in spectra are dependent on a number of factors, such as sample preparation and instrument geometry, peak positions are relatively insensitive to experimental details.

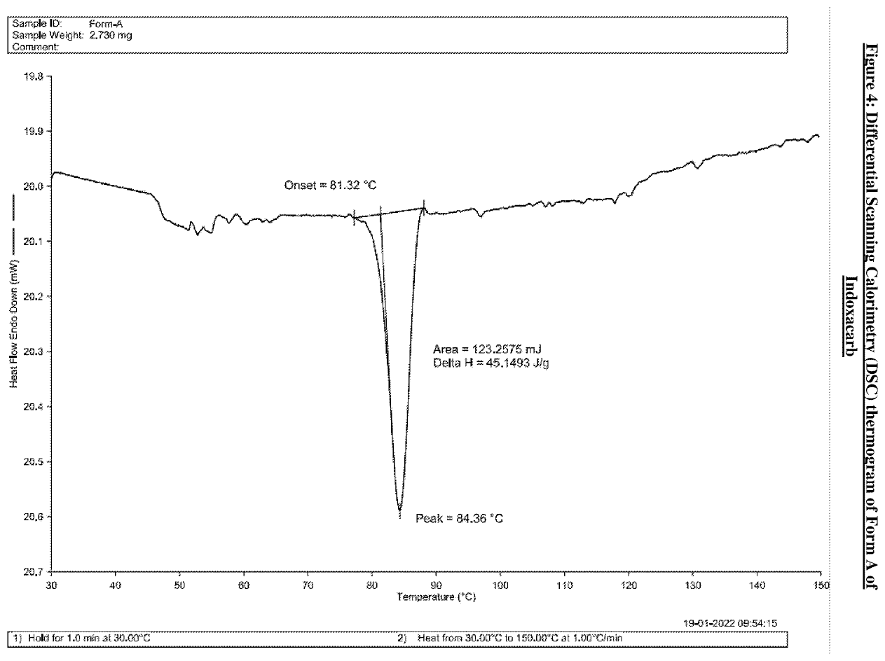

The reference solid state Form A of Indoxacarb also exhibits a Differential Scanning Calorimetry (DSC) thermogram substantially as shown in FIG. 4, which is characterized by a predominant endotherm peak at about between 80-90° C. as measured by Differential Scanning Calorimeter at a scan rate of 1° C. per minute.

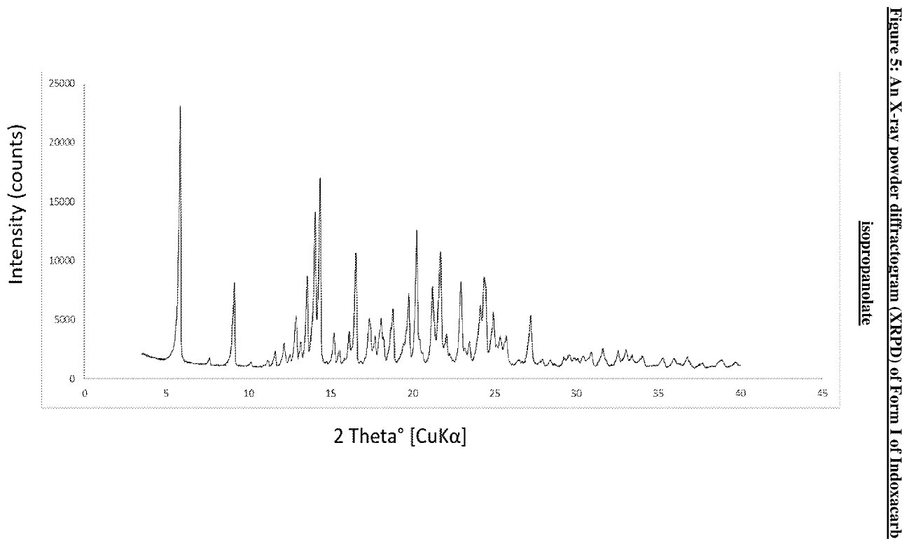

The present disclosure also includes a solid state form of Indoxacarb isopropanolate solvate designated as Form I. The Form I of Indoxacarb isopropanolate may be characterized by data selected from one or more of the following: an XRPD pattern having peaks at 5.8 degree 2-theta±0.2 degrees 2-theta; an XRPD pattern substantially as shown in FIG. 5; and combinations of these data, wherein by “substantially” it is meant that the reported peaks can vary by about ±0.2°. It is well known in the field of XRPD that while relative peak heights in spectra are dependent on a number of factors, such as sample preparation and instrument geometry, peak positions are relatively insensitive to experimental details.

Crystalline Form I of Indoxacarb isopropanolate solvate may be further characterized by the XRPD pattern having peak at 5.8 degree 2-theta±0.2 degrees 2-theta, and also having one, two or three additional peaks selected from 11.6, 14.1, 14.3, 16.5, 17.4, 18.8, 20.2, 21.7, 22.9 and 24.3 degrees 2-theta±0.2 degrees 2-theta. Crystalline Form I of Indoxacarb isopropanolate may be characterized by any combination of at least four peaks in an XRPD pattern substantially as shown in FIG. 5.

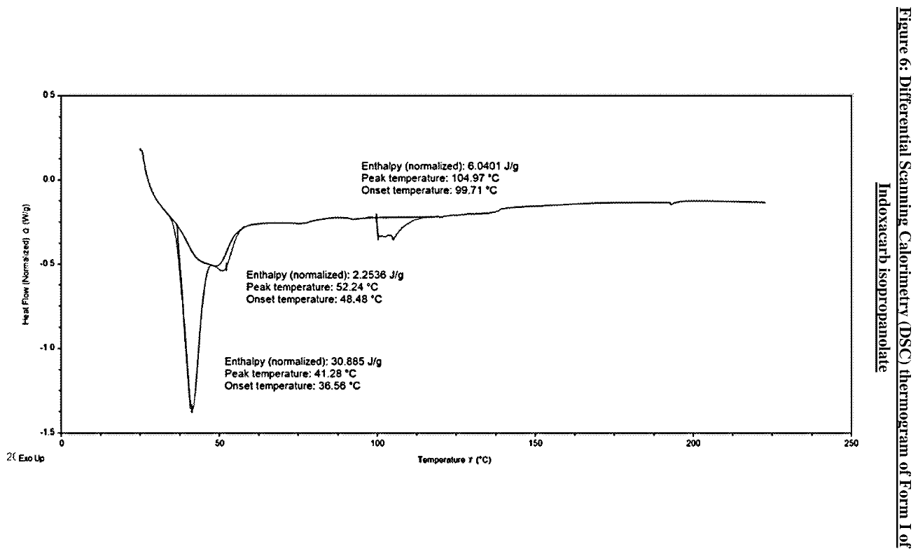

Crystalline Form I of Indoxacarb isopropanolate solvate also exhibits a Differential Scanning Calorimetry (DSC) thermogram substantially as shown in FIG. 6, which is characterized by a predominant desolvation endothermic peak at about between 40-50° C. as measured by Differential Scanning Calorimeter.

The present disclosure also provides the solid state forms of Indoxacarb of the present disclosure for use in the preparation of other solid state forms of Indoxacarb.

The present disclosure further encompasses process for preparing Indoxacarb solid state Form B as well as process for preparing Form I of Indoxacarb isopropanolate. The processes include starting from Form A of Indoxacarb, which was prepared following the process of the PCT publication WO2018178982 by recrystallization from Ethyl acetate and n-Heptane.

In an embodiment, the present disclosure covers process for preparing Indoxacarb solid state Form B which includes starting from Form A of Indoxacarb, and converting it to Form B of Indoxacarb, by a process, for example, including subjecting a solution of Form A of Indoxacarb in an alcohol solvent system followed by refluxing, cooling, filtering and drying under vacuum to obtain a solid form, which is Form B of Indoxacarb, wherein the alcohol solvent may be selected from a group comprising Isopropyl alcohol, 1-Propanol and isobutyl alcohol.

In another embodiment, the present disclosure covers process for preparing Form I of Indoxacarb isopropanolate which includes starting from Form A of Indoxacarb, and converting it to Form I of Indoxacarb isopropanolate, by a process, for example, including dissolving Form A of Indoxacarb in an isopropanol in high temperature, and after cooling subjecting the solution with additional solid Indoxacarb followed by stirring, filtering and drying to obtain a solid form, which is Form I of Indoxacarb isopropanolate solvate. Isolation of form I in isopropanol as solvent is preferred when the solvent is substantially free of water, preferably not more than 10%, more preferably not more than 5%, and most preferably not more than 2% of water.

In another embodiment, the present disclosure encompasses the above described solid state forms of Indoxacarb or its solvate for use in the preparation of agrochemical compositions and/or formulations, optionally for agronomic and non-agronomic uses, and a method of their use for controlling lepidopteron pests in both agronomic and non-agronomic environments.

In another embodiment, the present disclosure encompasses the use of the described solid state forms of Indoxacarb or its solvate for the preparation of agrochemical compositions and/or formulations.

The present disclosure further provides agrochemical compositions including one or more solid state forms of Indoxacarb or its solvate according to the present disclosure.

In yet another embodiment, the present disclosure encompasses agrochemical formulations including one or more of the described solid state forms of Indoxacarb or its solvate and at least one additional component selected from the group consisting of a surfactant, a solid diluent and liquid diluent and optionally an effective amount of at least one additional biologically active compound or agent.

In an embodiment, the present disclosure encompasses processes to prepare said agrochemical formulations of Indoxacarb or its solvate including one or more of the described solid state forms and at least one additional component selected from the group consisting of a surfactant, a solid diluent and liquid diluent and optionally an effective amount of at least one additional biologically active compound or agent.

In one embodiment, the solid state forms defined herein as well as the agrochemical compositions or formulations of the solid state forms of Indoxacarb or its solvate can be used as oxadiazine insecticide for controlling lepidopteron pests in both agronomic and non-agronomic environments.

In further embodiment, the present disclosure also encompasses methods for controlling lepidopteron pests comprising applying solid state forms of Indoxacarb or its solvate by blocking the sodium channels in the pest's nervous system, entering through the stomach and contact routes.

In yet another embodiment, the present disclosure also covers uses of the solid state forms of Indoxacarb or its solvate of the present disclosure, or at least one of the above agrochemical compositions or formulations for controlling lepidopteron pests in both agronomic and non-agronomic environments.

Having described the disclosure with reference to certain preferred embodiments, other embodiments will become apparent to one skilled in the art from consideration of the specification. The disclosure is further illustrated by reference to the following examples describing in detail the preparation of the solid forms and methods of use of the disclosure. It will be apparent to those skilled in the art that many modifications, both to materials and methods, may be practiced without departing from the scope of the disclosure.

### Analytical Methods

### Measurement of Form B

### X-Ray Powder Diffraction Method:

Powder X-ray Diffraction was performed on an X-Ray powder diffractometer Bruker Axs; CuK_{α}, radiation, (λ=1.54187 Å); zero background sample holders.

### Measurement Parameters:

Scan range: 3-40 degrees 2-theta
Spinner Rotation: 5 RPM
Time per step (scan speed): 0.7 s
Run time: 1 hour 32 sec
Sample holder: Zero background silicon plate
   Prior to analysis, the samples were gently ground using a mortar and pestle to obtain a fine powder. Optionally, silicon powder can be added in a suitable amount as internal standard in order to calibrate the positions of the diffractions. The ground sample was adjusted into a cavity of the sample holder and the surface of the sample was smoothed using a cover.

### Dsc Conditions:

System: Perkin Elmer 4000 DSC

### Conditions:

1. Hold for 1 min at 30° C.
2. Heat from 30° C. to 150° C. at scan rate 1° C./min.

### Measurement of Form I

### X-Ray Powder Diffraction Method:

X-ray powder diffraction (XRPD) analysis was performed by a Panalytical Empyrean powder diffractometer (Cu Kₐ radiation, 1=1.54178 Å) equipped with an X'Celerator linear detector and operated at V=40 kV, I=30 mA. Regular continues scans were run in a 2-theta range of 3.5÷40° with step equal to ˜0.0167°, time/step ˜40 sec, scan speed ˜0.053°/sec.

### DSC Conditions:

TA Instruments Discovery DSC 2500 equipped with a RC90 cooler.

### Conditions

The sample was heated from 25° C. to 225° C. at 10° C./min. Nitrogen was used as the furnace purge gas, at a flow rate of 50 cm³/min.

### EXAMPLES

The starting material, Form A of Indoxacarb was prepared following the process of the PCT publication WO2018178982 by recrystallization from Ethyl acetate and n-Heptane. The chemical purity of Form A of Indoxacarb is >99% and enantiomeric ratio (S:R) is 89.25/10.75.

New solid state forms of Indoxacarb were prepared according to the following procedure:

### Example 1: Preparation of Indoxacarb Form B

A round bottom flask with mechanical stirrer, thermoprobe, condenser, gland, stopper, etc. was arranged and 500 mL (5.0 vol.) Isopropyl alcohol (IPA) was charged into it at around 25° C. and 100.0 g of solid Form A of Indoxacarb was charged at the same temperature. The reaction mass was stirred for 5-10 mins and the temperature was raised to Reflux 80-82° C. and was maintained at that temperature for 30 mins. The reaction mass was cooled to 30-35° C. for 2 hrs and was filtered under vacuum at 30-35° C. The wet cake was treated with 50 ml IPA for washing and the mother liquid was stirred for 4 h at 20-25° C. The reaction mass was filtered, and the solid product was washed with IPA (0.5 vol.) and suck dried under vacuum for 30 min. The product is then dried in vacuum oven under vacuum (700 mm/Hg) at 30-35° C. for 24 hrs. After completion of drying the solid product was unloaded in 50% yield (50.2 g). Solid was then gently grounded with spatula and analysed by XRD and DSC.

The chemical purity of Form B of Indoxacarb is >99% and enantiomeric ratio (S:R) is 95.5/4.4.

### Example 2: Preparation of Indoxacarb Isopropanolate Solvate Form I

100.0 g of Indoxacarb form A was dissolved at 70° C. in 400 g IPA (Isopropyl alcohol). The Indoxacarb solution was cooled to 20° C. for 2 hours and stirred at the same temperature for 1 hour. 0.75 gr of solid Indoxacarb (form A) was charged at the same temperature. The reaction mass was stirred for 3.5 hours at that temperature. The reaction mass was cooled to 12.5° C. for 3.5 hours and stirred at the same temperature for 7 hours. The reaction mass was cooled to −5° C. for 7 hours and stirred at the same temperature for 1.5 hours. The obtained crystalline material was filtered out, washed with IPA then dried to afford 60.0 g solid. Solid was then gently grounded with spatula and analysed by XRD and DSC.

The solid contains about 12% isopropanol and displays a DSC characterized by an endothermic peak at about 40-50° C. due to isopropanol desolvation.
The chemical purity of Form I of Indoxacarb Isopropanolate solvate is found to be 96% and enantiomeric ratio (S:R) is 9:1.

1. A Crystalline Form B of Indoxacarb, which is characterized by data selected from one or more of the following:
i. an XRPD pattern having peaks at 13.5, 13.8, 16.2, 21.2, and 21.4 degrees 2-theta±0.2 degrees 2-theta;
ii. an XRPD pattern substantially as shown in FIG. 1.

2. The crystalline form of Indoxacarb according to claim 1, which is characterized by an XRPD pattern having peaks at 13.5, 13.8, 16.2, 21.2, and 21.4 degrees 2-theta±0.2 degrees 2-theta, and also having one, two or three additional peaks selected from 7.9, 19.4 and 23.9 degrees 2-theta±0.2 degrees 2-theta.

3. A Crystalline Form I of Indoxacarb isopropanolate, which is characterized by data selected from one or more of the following:
i. an XRPD pattern having peak at 5.8 degree 2-theta±0.2 degrees 2-theta;
ii. an XRPD pattern substantially as shown in FIG. 5.

4. The Crystalline Form I of Indoxacarb isopropanolate according to claim 3, which is characterized by an XRPD pattern having peak at 5.8 degree 2-theta±0.2 degrees 2-theta, and also having one, two or three additional peaks selected from 11.6, 14.1, 14.3, 16.5, 17.4, 18.8, 20.2, 21.7, 22.9 and 24.3 degrees 2-theta±0.2 degrees 2-theta;

5. An agrochemical composition comprising a crystalline form according to any one of claims 1-4.

6. An agrochemical formulation comprising a crystalline form according to any one of claims 1-4 or an agrochemical composition of claim 5, and at least one agrochemically acceptable excipient.

7. A method for controlling lepidopteron pests by using a crystalline form according to any one of claims 1-4, an agrochemical composition according to claim 5, or an agrochemical formulation according to claim 6, as an insecticide in both agronomic and non-agronomic environments.

8. A process of preparation of the crystalline form of Indoxacarb according to claim 1, comprising subjecting a solution of Form A of Indoxacarb in an alcohol solvent system followed by refluxing, cooling, filtering and drying under vacuum to obtain a solid form, which is Form B of Indoxacarb.

9. The process of preparation of the crystalline form of Indoxacarb according to claim 8, wherein the alcohol solvent is selected from a group comprising Isopropyl alcohol, 1-Propanol and isobutyl alcohol.

10. The process of preparation of the crystalline form of Indoxacarb according to any of claims 8-9, wherein the alcohol solvent is Isopropyl alcohol.

11. A process of preparation of the crystalline form of Indoxacarb isopropanolate solvate according to claim 3, comprising dissolving Form A of Indoxacarb in Isopropyl alcohol in high temperature, and after cooling subjecting the solution with additional solid Indoxacarb followed by stirring, filtering and drying to obtain a solid, which is Form I of Indoxacarb isopropanolate solvate.

12. A process of preparation of the crystalline form I of Indoxacarb isopropanolate solvate according to claim 3, comprising dissolving Form A of Indoxacarb in Isopropyl alcohol substantially free of water.
